(19) United States
(12) Patent Application Publication
(10) Pub. No.: US 2013/0084276 A1
(43) Pub. Date: Apr. 4, 2013

(54) BIOMARKERS OF CARDIOVASCULAR DISEASE INCLUDING LRG

(71) Applicants: Chris Watson, Dublin (IE); Mark Ledwidge, Dublin (IE); Kenneth McDonald, Dumblin (IE); John Baugh, Dublin (IE)

(72) Inventors: Chris Watson, Dublin (IE); Mark Ledwidge, Dublin (IE); Kenneth McDonald, Dumblin (IE); John Baugh, Dublin (IE)

(73) Assignees: The Heartbeat Trust Limited; University fo College Dublin, National University of Ireland

(21) Appl. No.: 13/575,276

(22) PCT Filed: Jan. 26, 2011
(86) PCT No.: PCT/EP2011/051088

§ 371 Date: Dec. 11, 2012

(30) Foreign Application Priority Data
Jan. 26, 2010 (IE) 2010/0036

(51) Int. Cl. G01N 33/68

(52) U.S. Cl. CPC G01N 33/6893
USPC 424/94.6; 435/7.1; 530/387.5; 536/24.31; 435/6.12; 506/16; 506/18; 514/16.4; 506/9; 435/7.92

(57) ABSTRACT

The invention relates to differentially expressed disease - associated proteins that have potential to identify patients with cardiovascular disease including ventricular dysfunction and heart failure and the potential to predict heart failure in patients. In particular, the invention relates to the use of a panel of biomarkers in the diagnostic and prognostic evaluation of cardiovascular patients. One of the markers is Leucine-rich alpha-2-glycoprotein (LRG).

### FIELD OF THE INVENTION

The invention relates to differentially expressed disease-associated proteins that have potential to identify patients with cardiovascular disease including ventricular dysfunction and heart failure and the potential to predict heart failure in patients. In particular, the invention relates to the use of a panel of biomarkers in the diagnostic and prognostic evaluation of cardiovascular patients.

### BACKGROUND TO THE INVENTION

While therapies for heart failure (HF) have improved, it is clear that effective prevention strategies will have the most important impact on the epidemiology of this syndrome. In this regard, it is essential to complement effective risk factor control with the earliest possible identification of those at risk of progressive ventricular dysfunction and HF. Hypertensive heart disease (HHD) is a well described example of progressive ventricular dysfunction. Currently, it is not known why a certain proportion of patients with hypertension develop diastolic dysfunction (DD) and others do not. Furthermore the early diagnosis of the transition to HF is critical and yet can be clinically challenging. A better understanding of the pathophysiological signals at play may allow for more precise diagnostic tests to define the onset of HF. This natural history is not simply explained by the degree of blood pressure control and likely involves complex disease mechanisms still not fully understood. The ability to identify which asymptomatic hypertensive patients will acquire a more detrimental disease phenotype would have a significant impact on treatment strategies and disease monitoring for this prevalent and important disease.

B-type natriuretic peptide(s) (BNP) have been shown by us and others to effectively identify various stages of DD in HHD and also to be of prognostic importance in established HFThe inventors have determined that elevated levels of this peptide in a hypertensive population identifies a more worrying natural history and that analysis of coronary sinus (CS) serum in this population may provide further insight into early disease recognition, disease stimuli and potential therapeutic interventions. The CS serum proteome of a subset of asymptomatic hypertensive patients that were stratified according to BNP levels using two-dimensional difference gel electrophoresis (2D-DIGE) was examined. This sensitive screening tool that enables careful dissection of the serum proteome identified a number of molecules as being differentially -expressed in asymptomatic patients with elevated BNP and at risk of developing heart failure. 2D-DIGE is also capable of indicating post-translational modifications based on isoelectric point (pi) shifting.

Leucine-rich alpha-2-glycoprotein (LRG) was identified in 1977 as a trace component of human serum (1) and resolution of the primary structure in 1985 indicated that it existed as a single polypeptide chain of approximately 45 kDa (2). The precise function of LRG has yet to be defined but evidence to date suggests that it is associated with inflammatory responses and neutrophilic differentiation as well as cellular responses to the profibrotic cytokine TGFβ. However, recent studies in patients with Hepatitis C have shown that LRG expression in patient serum decreases with increasing severity of fibrotic change in diseased livers. Thus it is difficult to predict how expression levels of given proteins will change in particular disease states and the exact role of LRG in fibrosis.

A growing body of data suggests that myocardial fibrosis is a central abnormality in the pathogenesis of HHD, DD and the development of HF. The factors driving myocardial fibrosis are yet to be fully elucidated but likely involve an exaggerated inflammatory response.

Heart Failure (HF) is a prevalent and growing disease, which is preceded by ventricular dysfunction. Prevention of HF is an important strategy as we attempt to control the growing epidemic. In this regard, natriuretic peptides have shown promise due to their ability to discriminate various stages of ventricular dysfunction. Furthermore, natriuretic peptide measurement is the guideline biochemical standard for the diagnosis of HF.

However, the utility of natriuretic peptides is limited by significant biological variability and emerging information on the dependence of this protein on non-cardiac influences such as beta blocker use, BMI, renal function and age.

Optimal prevention of HF necessitates identification of at-risk patients prior to the development of ventricular dysfunction. Early and accurate diagnosis of HF requires improved diagnostic biomarkers and likely a multi-biomarker approach. The diagnosis of HF is a complex clinical diagnosis requiring expert clinical assessment and objective evidence of cardiac dysfunction. Currently the gold standard for objective evidence of cardiac dysfunction is echocardiography. However, echocardiography is expensive and requires specialist technical expertise and equipment. Other imaging techniques such as cardiac MRI can provide objective evidence, but also require specialist expertise, expensive equipment and are limited in availability.

Therefore, identification of other biomarkers and their use in isolation or with natriuretic peptides may provide a more accurate detection of sub-clinical disease and indeed prediction of progression, outcome or response to treatment.

B-type natriuretic peptide(s) (BNP) have been shown by us and others to effectively identify various stages of DD in HHD and also to be of prognostic importance in established HF. Recently, biochemical evidence of ventricular dysfunction using B-type natriuretic peptide (BNP) has become accepted in international guidelines for the establishment of this objective evidence of ventricular dysfunction. Diagnostic biomarkers can be evaluated in point-of-care assays at lower cost and facilitate the early and accurate diagnosis of heart failure. This facilitates the initiation of appropriate therapy that can prolong life and reduce morbidity. Therapies associated with these peptides have been developed, although their exact role in care of heart failure has not been defined.

Because of the problems with the availability, cost and technical expertise of the imaging modalities, it is not surprising that the majority of HF in the community remains undiagnosed. Diagnostic biomarkers, particularly when used in point-of-care settings are cheaper and more widely applicable to clinical settings. However, the gold standard diagnostic biomarker in HF, currently BNP is limited by significant biological variability and emerging information on the dependence of this protein on non-cardiac influences such as beta blocker use, BMI, renal function and age. This can confound the effectiveness of this biomarker. Furthermore, while diagnostic biomarkers point to potential therapies and BNP analogues have been commercialised, their exact role in therapy is not well defined.

This invention has shown that the biomarkers can be used to predict heart failure (HF) in patients, including Diastolic Heart Failure (DHF). As the prevalence of heart failure syndromes increase, the importance of heart failure with preserved ejection fraction (HF-PEF) and DHF is becoming increasingly accepted. Recent studies have shown that 30-55% of HF patients hospitalized with acute decompensation have normal or near normal left ventricular ejection fraction (LVEF). As the predominant form of HF in women, hypertensives and the elderly, the relative importance of HF-PEF is set to rise even further in the future. To best curb this growing epidemic, increased focus needs to be placed on disease prevention in those at risk; in particular, those with asymptomatic left ventricular diastolic dysfunction (LVDD), a recognized prequel to the development of HF-PEF/DHF. ⁶ This approach has proven effective in patients with asymptomatic left ventricular systolic dysfunction where pharmacological intervention can reduce the incidence of HF and improve prognosis.

The Rochester Epidemiology Project found prevalence rates for LVDD in the general community of 21%, 7% and 1% for mild, moderate and severe dysfunction respectively. High-risk groups with even greater prevalence rates included the elderly and those with hypertension, diabetes mellitus (DM), coronary artery disease, obesity or systolic dysfunction; for example, amongst those over 65 yrs of age with hypertension or CAD, 48% had mild LVDD while 17% had moderate or severe LVDD. Furthermore, the prognostic importance of LVDD was demonstrated in a multivariate analysis controlled for age, sex, and LVEF, whereby mild LVDD and moderate or severe LVDD were predictive of all-cause mortality (hazard ratios of 8 and 10 respectively). Given that patients with LVDD are at particular risk of experiencing cardiovascular events such as HF, an analysis of the natural history of LVDD along with a stratification of risks underlying LVDD progression is warranted. There is thus a need to provide improved diagnostic and prognostic means for cardiovascular disease.

### Object of the Invention

Heart failure prevention strategies require biomarkers that predict disease manifestation. It is thus an object of the invention to identify differentially expressed disease-associated proteins that can identify patients with cardiovascular disease including ventricular dysfunction and heart failure. A further object of the invention is to provide markers and methods for predicting heart failure in a patient. Another object is to provide biomarkers for the identification of left ventricular diastolic dysfunction. It is a further object of the invention to identify novel biomarkers which can be used in methods and diagnostic kits and assays for the identification of patients at risk of developing cardiovascular disease. A still further object is to provide a simple and reliable method for the identification of patients at risk of developing cardiovascular disease and in particular one which can be carried out on a blood sample or at the point of care. A still further object is to provide means of identifying therapeutic agents for the prevention and treatment of cardiovascular disease.

### SUMMARY OF THE INVENTION

According to the present invention there is provided use of Leucine-rich alpha-2-glycoprotein, as a biomarker for cardiovascular disease.

LRG may be used in conjunction with at least one protein selected from the group comprising complement factor i precursor, pigment epithelium—derived factor precursor, serum paraoxonase/arylesterase 1, Beta-2-glycoprotein (Fib), apolipoprotein a-iv precursor, apolipoprotein a-i precursor, Complement C3 (Fib), tetranectin precursor, Serum amyloid P-component, complement c1s subcomponent precursor, vitamin d-binding protein precursor, isoform 1 of gelsolin precursor, Isoform Migration stimulation factor FN70 of Fibronectin, Zinc-alpha-2-glycoprotein, Clusterin and transthyretin precursor as markers for cardiovascular disease.

According to another aspect of the present invention there is provided use of at least one protein selected from the group comprising complement factor i precursor, pigment epithelium—derived factor precursor, serum paraoxonase/arylesterase 1, Beta-2-glycoprotein (Fib), apolipoprotein a-iv precursor, apolipoprotein a-i precursor, Leucine-rich alpha-2-glycprotein, Complement C3 (Fib), tetranectin precursor, Serum amyloid P-component, complement c1s subcomponent precursor, vitamin d-binding protein precursor, isoform 1 of gelsolin precursor, Isoform Migration stimulation factor FN70 of Fibronectin, Zinc-alpha-2-glycoprotein, Clusterin and transthyretin precursor, as a biomarker for cardiovascular disease.

In a preferred embodiment the biomarker is leucine-rich alpha-2-glycoprotein (LRG). The invention also provides a method of determining the risk of developing cardiovascular disease in a patient comprising taking a blood sample from a patient and determining the level at least one protein selected from the group comprising complement factor i precursor, pigment epithelium—derived factor precursor, serum paraoxonase/arylesterase 1, Beta-2-glycoprotein (Fib), apolipoprotein a-iv precursor, apolipoprotein a-i precursor, Leucine-rich alpha-2-glycprotein, Complement C3 (Fib), tetranectin precursor, Serum amyloid P-component, complement c1 s subcomponent precursor, vitamin d-binding protein precursor, isoform 1 of gelsolin precursor, Isoform Migration stimulation factor FN70 of Fibronectin, Zinc-alpha-2-glycoprotein, Clusterin and transthyretin precursor, in the sample, an elevated or reduced level of the at least one protein, in comparison to a control indicating a pre-disposition to develop cardiovascular disease. The control value may be obtained from a patient who is known not to have cardiovascular disease. The presence or an increased level compared to a control of some markers may be indicative of disease whilst the absence or a decreased level compared to a control of other markers may be indicative of disease. This information is found under the heading Fold Change in Table 1.

The method may comprise determining the level of at least 2 proteins from the group, or at least 3 proteins, or at least 4 proteins, or at least 5 proteins, or at least 6 proteins form the group. In other embodiments the invention provides determining the level of all 17 proteins from the group, or at least 10 proteins from the group.

The blood sample may be a coronary sinus or peripheral blood sample. The blood sample may be a capillary blood samples or a finger prick sample.

In another aspect the invention provides a diagnostic assay for determination of the risk of developing cardiovascular disease in a patient comprising an antibody against at least one protein selected from the group comprising complement factor i precursor, pigment epithelium—derived factor precursor, serum paraoxonase/arylesterase 1, Beta-2-glycoprotein (Fib), apolipoprotein a-iv precursor, apolipoprotein a-i precursor, Leucine-rich alpha-2-glycprotein, Complement C3 (Fib), tetranectin precursor, Serum amyloid P-component, complement c1 s subcomponent precursor, vitamin d-binding protein precursor, isoform 1 of gelsolin precursor, Isoform Migration stimulation factor FN70 of Fibronectin, Zinc- alpha-2-glycoprotein, Clusterin and transthyretin precursor, or a nucleotide probe for at least one protein selected from the group or a portion thereof, the probe being DNA, RNA or cDNA.

The assay may be selected from a real-time PCR assay, a micro-array assay, a histochemical assay or an immunological assay. For LRG assays cytochrome C may be used as a capturing ligand for building an ELISA. All such assays are well known to those of skill in the art. Where the assay is a histochemical assay, the antibody may be labelled with a suitable label. Suitable labels include coloured labels, fluorescent labels and radioactive labels.

The invention further provides a method of identifying a therapeutic agent capable of preventing or treating cardiovascular disease, comprising testing the ability of the potential therapeutic agent to reduce or enhance the expression of at least one protein selected from the group comprising complement factor i precursor, pigment epithelium—derived factor precursor, serum paraoxonase/arylesterase 1, Beta-2-glycoprotein (Fib), apolipoprotein a-iv precursor, apolipoprotein a-i precursor, Leucine-rich alpha-2-glycprotein, Complement C3 (Fib), tetranectin precursor, Serum amyloid P-component, complement c1s subcomponent precursor, vitamin d-binding protein precursor, isoform 1 of gelsolin precursor, Isoform Migration stimulation factor FN70 of Fibronectin, Zinc-alpha-2-glycoprotein, Clusterin and transthyretin precursor, in a cell or cell line, or an animal or human test subject.

Further provided is a method of identifying a therapeutic agent capable of preventing or treating cardiovascular disease, comprising contacting at least one protein selected from the group comprising complement factor i precursor, pigment epithelium—derived factor precursor, serum paraoxonase/arylesterase 1, Beta-2-glycoprotein (Fib), apolipoprotein a-iv precursor, apolipoprotein a-i precursor, Leucine-rich alpha-2-glycprotein, Complement C3 (Fib), tetranectin precursor, Serum amyloid P-component, complement c1s subcomponent precursor, vitamin d-binding protein precursor, isoform 1 of gelsolin precursor, Isoform Migration stimulation factor FN70 of Fibronectin, Zinc-alpha-2-glycoprotein, Clusterin and transthyretin precursor, with a putative therapeutic agent and determining if the agent modulates the activity of the protein when compared with a control.

Also provided is a method of prevention or treatment of cardiovascular disease comprising administering to a patient in need of such treatment, an inhibitor of or an agent which can silence, or an agent which can enhance the expression of at least one protein selected from the group comprising complement factor i precursor, pigment epithelium—derived factor precursor, serum paraoxonase/arylesterase 1, Beta-2-glycoprotein (Fib), apolipoprotein a-iv precursor, apolipoprotein a-i precursor, Leucine-rich alpha-2-glycprotein, Complement C3 (Fib), tetranectin precursor, Serum amyloid P-component, complement c1s subcomponent precursor, vitamin d-binding protein precursor, isoform 1 of gelsolin precursor, Isoform Migration stimulation factor FN70 of Fibronectin, Zinc-alpha-2-glycoprotein, Clusterin and transthyretin precursor.

The invention also provides a solid support onto which at least one protein selected from the group comprising complement factor i precursor, pigment epithelium—derived factor precursor, serum paraoxonase/arylesterase 1, Beta-2-glycoprotein (Fib), apolipoprotein a-iv precursor, apolipoprotein a-i precursor, Leucine-rich alpha-2-glycprotein, Complement C3 (Fib), tetranectin precursor, Serum amyloid P-component, complement c1s subcomponent precursor, vitamin d-binding protein precursor, isoform 1 of gelsolin precursor and transthyretin precursor, or antibodies raised against them, or nucleic acid probes for the proteins, have been fixed.

All of the above methods, assays, supports and kits may comprise determining the level of at least 2 proteins from the group, or at least 3 proteins, or at least 4 proteins, or at least 5 proteins, or at least 6 proteins form the group. In other embodiments the invention provides determining the level of all 17 proteins from the group, or at least 10 proteins from the group. Where the reduction or enhancement of protein expression is described in the specification in relation to methods or assays as described above it will be apparent to the skilled person in the context of the particular assay or method whether the protein in question should be enhanced or reduced. This information is found under the heading Fold Change in Table 1. For example if a protein shows reduced expression a method of treatment of cardiovasculer disease would involve enhancing expression of the protein and a method of identifying therapeutic agents would involve finding an agent which could enhance expression of the protein in question.

### BRIEF DESCRIPTION OF THE DRAWINGS

FIG. 1. Differential expression of leucine-rich alpha-2-glycoprotein (LRG) in asymptomatic hypertensive patients. Two-dimensional difference gel electrophoresis (2D-DIGE) gel highlighting the differentially expressed protein spots ID 0416 and ID 0442 (spot 1 and spot 2, respectively), later identified using mass spectrometry as LRG (A). Protein spots 3, 4, and 5 were also discovered to be LRG but were not differentially expressed between the two groups. Verification of increased coronary sinus (CS) LRG in patients with elevated B-type natriuretic peptide (BNP) (n=6, range 11.3:28.1 μg/ml) versus low BNP (n=5, range 9.5:17.0 μg/ml) in the 11 samples analyzed with 2D-DIGE (B) using an LRG-specific ELISA. The relationship between CS levels of LRG (n=40, range 6.1:28.1) and B-type natriuretic peptide (BNP) in asymptomatic hypertensive patients (C) using ELISA based approach. and in an extended cohort, totaling 40 (C).

FIG. 2: Correlation between B-type natriuretic peptide (BNP) and leucine-rich alpha-2-glycoprotein (LRG) in patients with left ventricular diastolic dysfunction (LVDD) and HF (A) analysed using ELISA based methodologies. Analysis of LRG in patients with asymptomatic LVDD (n=32, range 25.2:43.5), diastolic heart failure (DHF) (n=26, range 27.4:47.8), and systolic heart failure (SHF) (n=47, range 25.5:59.5) (B). ROC curve comparing LRG and BNP in identifyingpredicting heart failure (n=73 HF vs. n=72 no-HF, AUC 0.86, 0.85 respectively, both p<0.001) (C).

FIG. 3: Correlation between coronary sinus (CS) leucine-rich alpha-2-glycoprotein (LRG) and CS tumor necrosis factor alpha (TNFα) and CS interleukin (IL) 6 in the serum of asymptomatic hypertensive patients (n=40, TNFα range 2.0:5.9 ,IL-6 range 0.7:9.5) using an ELISA approach (A). Evidence of LRG mRNA expression in 4 atrial cardiac tissue samples, primary human coronary artery endothelial cells (HCAEC), primary human ventricular cardiac fibroblast cells (HVCF), and neutrophils which served as a positive control (B) as indicated by RT-PCR analysis. Correlation between LRG and transforming growth factor beta receptor 1 (TGFβR1) and alpha smooth muscle actin (ASMA) cardiac tissue mRNA (n=40) (C). Representative image of right atrial biopsy tissue used in this study. Tissue was stained with hematoxylin and eosin for morphological assessment (D).

Immunohistochemical evidence of LRG protein expression within myocardial tissue (E). Cardiac myocytes (examples indicated with purple arrows) exhibited positive membranous (black arrow) and cytoplasmic immunostaining (grey arrow). Cardiac fibroblast cells (examples indicated with green arrows), appear to be negative for LRG protein expression as indicated by immunohistochemistry. Images presented are original magnification x 20 and x40.

### DETAILED DESCRIPTION OF THE INVENTION

Methods

Patient Recruitment and Sample Collection

Patients

All subjects gave written informed consent to participate in the study. The Ethics Committee at St. Vincent's University Hospital approved the study protocols which conformed to the principles of the Helsinki Declaration. The study populations reported here consist of 145

Caucasian patients divided into four sub-groups; asymptomatic Hypertension (n=40) (Table 1), asymptomatic Left Ventricular Diastolic Dysfunction (LVDD) (n=32), Diastolic HF (DHF) (n=26), and Systolic HF (SHF) (n=47) (Table 2) while cardiac biopsies were taken from 40 patients undergoing heart surgery. The initial discovery phase of the study utilized the asymptomatic hypertensive patients. The remaining patient cohorts, LVDD, DHF, and SHF, were used in the validation arm of the study. These various cohorts represent the different phases of the natural history of hypertensive heart disease.

Asymptomatic hypertensive patients had normal systolic function with ejection fraction (LVEF) ≧50%. The diagnosis of DHF was based on the presence of all of the following criteria; one hospitalization for proven Class IV HF (confirmed by a consultant cardiologist) and continued symptoms of at least NYHA Class II, LVEF≧45% with Doppler-echocardiographic abnormalities of DD without significant evidence of valvular disease. The diagnosis of SHF was based on symptoms and clinical signs of HF and LVEF ≦45%. Exclusion criteria included patients with unstable symptoms of HF, those with renal insufficiency (serum creatinine >130 mmol) and those with clinical evidence of infection. Patients who underwent recent surgery or had recent physical trauma (<6 months) were also excluded. All patients underwent appropriate clinical and laboratory evaluation to identify exclusion criteria and suitability for this study.

For the study of patients with LVDD progression, inclusion criteria for the present study were age ≦40 years and at least one risk factor for LVDD including; hypertension (medicated for ≦1 month), hypercholesterolemia, obesity, coronary artery disease (confirmed by angiography) or DM. Exclusion criteria included significant LVDD at baseline (LAVI>32 mls/m²), known asymptomatic LV systolic dysfunction [defined as left ventricular ejection fraction (LVEF) <50% by echocardiography], previous or current heart failure, atrial fibrillation or any valvular heart disease of more than minor severity.

LVDD Progression Study Design

This was a prospective cohort study involving a population of 228 patients with risk factors for LVDD. Patients were randomly selected from the STOP-HF study [NCT00921960], an ongoing prospective randomized controlled trial recruiting asymptomatic ambulatory individuals with risk factors for left ventricular dysfunction from 50 primary care clinics in Dublin and south east Ireland. The study compared patients with echocardiographic evidence of LVDD progression at annual review to those without evidence of progression. The definition of LVDD progression was based upon left atrial volume index (LAVI) which is now considered a robust, continuous echocardiographic measure of LVDD that has relative load independence (3,4). Progressors in diastolic dysfunction were identified as those having a change in LAVI (ΔLAVI) ≦3.5 mls/m² from an initial LAVI between 20 and 32 mls/m². Non-progressors were similarly identified as those having ΔLAVI<3.5 mls/m² from an initial LAVI between 20 and 32 mls/m². The distribution of baseline LAVI measurements was similar in both groups.

Clinical Assessment

At each visit, a full physical examination, NYHA functional class assessment, resting electrocardiography and fasting phlebotomy were performed by a blinded observer. Physical examination included assessment of waist circumference, body mass index calculation (using the Mosteller formula), heart rate measurement and blood pressure measurement. Peripheral venous blood samples were tested for levels of creatinine, fasting glucose and lipids and brain natriuretic peptide (BNP).

Doppler-Echocardiography

Doppler-echocardiography assessment was performed by a single blinded operator according to the guidelines laid down by the American Society of Echocardiography. Restrictive-like filling patterns are severe forms of diastolic dysfunction and are defined from Doppler Echocardiographic indices as deceleration time <160 milliseconds with ≧1 of the following: left atrial size >5 cm, E/A ratio >1.5, and intraventricular relaxation time <70 milliseconds.

Left ventricular ejection fraction (LVEF) was calculated by the Teichholz method. Left ventricular mass was calculated using the Devereux method and was indexed to body surface area. Relative wall thicknesses were calculated by dividing the thickness of the septum and posterior wall by the left ventricular end-diastolic radius. Left atrial volume was calculated using the biplane area length method and was also indexed to body surface area. Left ventricular filling pressures were non-invasively assessed by E/e′. Tissue Doppler measurements were taken at the lateral mitral annulus.

Coronary Sinus and Peripheral Blood Sampling

CS sampling was performed in the asymptomatic hypertensive population during routine clinically indicated cardiac catheterization for suspected ischemic chest pain. These patients did not have unstable angina. Peripheral venous blood samples were obtained during clinical assessment. Serum samples were obtained following centrifugation at 2500 g for 10 minutes at 4° C. Samples were aliquoted and stored at −80° C. until required. All steps from collection to storage were carried out at 4° C. where possible and within a time-frame of less than 30 minutes. Each serum sample underwent no more than 3 freeze/thaw cycles prior to analysis.

Sample Preparation Prior to 2D-DIGE

Depletion of CS Serum

Eleven CS serum samples from asymptomatic hypertensive patients (6 high BNP ≧100 pg/ml and 5 low BNP <100 pg/ml) were affinity depleted of the six most abundant serum proteins; albumin, immunoglobulin G, immunoglobulin A, transferrin, heptoglobin, and anti-trypsin. These proteins constitute approximately 85-90% of the serum proteome. This was to enhance the sensitivity of downstream analysis and potentially reveal the less abundant pathologically relevant proteins of interest. This was achieved using the human Multiple Affinity Removal System, MARS Hu-6 column (Agilent Technologies), as previously described (18)

Sample Labeling with CyDyes

Depleted serum samples were precipitated using ice cold acetone and re-suspended in DIGE Lysis Buffer (9.5 M Urea, 2% CHAPS, 20 mM Tris, pH 8.5). Samples were cleaned up and concentrated using the Ettan 2-D Clean-up Kit (GE Healthcare), and quantified using the Bradford assay. CS serum samples were adjusted to a concentration of 2 μg/μl using DIGE Lysis Buffer and labeled with CyDye DIGE Fluors (minimal dyes) for Ettan DIGE (GE Healthcare). For each sample, 40 μg of serum protein was labeled with 320 pmol Cy5. In addition, an internal standard was generated by pooling together 40pg of each serum and labeling with Cy3.

2D-DIGE and Image Analysis

First Dimension Isoelectric Focusing

Proteins were separated according to charge (pl) under denaturing conditions using immobilised pH gradients (IPG). For each sample to be analyzed, 40 μg of Cy3 labeled internal control was combined with 40 μg of Cy5 labeled serum sample and mixed with 2×

Sample Buffer (9.5 M Urea, 2% CHAPS, 2% Dithiothreitol (DTT), 1.6% Pharmalyte). Rehydration Buffer (8 M Urea, 0.5% CHAPS, 0.2% DTT, 0.2% Pharmalyte) was added to the sample and overlaid onto a pH 4-7, 24 cm IPG Immobiline™ DryStrip (GE Healthcare). Passive in-gel rehydration was allowed to occur overnight in the dark. The IPG strip rehydrated with the sample was focused in the first dimension using an Ettan IPGphor3 Isoelectric Focusing unit (GE Healthcare).

Second Dimension SDS-PAGE

Proteins were separated according to their molecular weight under denaturing conditions. IPG strips were equilibrated using two equilibration buffers (6 M Urea, 50 mM TrisCl pH 8.8, 30% (v/v) Glycerol, 2% (w/v) SDS, 1% (w/v) DTT) for 15 minutes followed by (6 M Urea, 50 mM TrisCl pH 8.8, 30% (v/v) Glycerol, 2% (w/v) SDS, 2.5% (w/v) iodoacetamide) for 15 minutes. Equilibrated IPG strips were added to 12% SDS-PAGE gels that were cast using low fluorescent plates. Gels were ran in a PROTEAN Plus Dodeca Cell tank (Bio-Rad) using 1× SDS Electrophoresis Buffer (25 mM Tris, 192 mM Glycine, 0.1% (w/v) SDS) at 1 Watt per gel, while being maintained at 15° C. and kept in the dark.

Following separation in the second dimension, gel cassettes were scanned using a Typhoon 9410 Variable Mode Imager scanner (GE Healthcare) at a resolution of 100 μM for quantitative computer image analysis with Progenesis PG240 software (Nonlinear Dynamics). Following software alignment of the 2D-DIGE images, normalization and statistical analysis, differentially expressed protein spots were identified.

Mass Spectrometry and Peptide Identification

Preparative 2D-gels loaded with 300 μg of depleted CS serum protein were generated for mass spectrometry identification of differentially expressed proteins. 2D-gels were fixed and silver stained using PlusOne Silver Stain Kit (GE Healthcare) according to the manufacturer's instructions. Protein spots of interest were isolated from the 2D-gels, de-stained, and trypsin digested. All samples were run on a Thermo Scientific LTQ ORBITRAP XL mass spectrometer connected to an Exigent NANO LC.1DPLUS chromatography system incorporating an auto-sampler. Tryptic peptides were resuspended in 0.1% formic acid. Each sample was loaded onto a Biobasic C18 Picofrit™ column (100 mm length, 75 μm ID) and was separated by an increasing acetonitrile gradient, using a 25 min reverse phase gradient (2-50% acetonitrile for 10 min) at a flow rate of 30 nL min⁻¹. The mass spectrometer was operated in positive ion mode with a capillary temperature of 200° C., a capillary voltage of 9V, a tube lens voltage of 100V and with a potential of 1800V applied to the frit. All data was acquired with the mass spectrometer operating in automatic data dependent switching mode. A high resolution MS scan was performed using the Orbitrap to select the 5 most intense ions prior to MS/MS analysis using the Ion trap.

The raw mass spectral data was analysed using Bioworks Browser 3.3.1 SP1, a proteomics analysis platform. All MS/MS spectra were sequence database searched using the algorithm TurboSEQUEST. The MS/MS spectra were searched against a redundant Human Swissprot database. The following search parameters were used: precursor-ion mass tolerance of 100 ppm, fragment ion tolerance of 1.0 Da with methionine oxidation and cysteine carboxyamidomethylation specified as differential modifications and a maximum of 2 missed cleavage sites allowed.

Serum Biomarker Assessment

BNP was quantified using a Triage meter BNP assay (Biosite Inc.). Serum levels of LRG were quantified using a Human LRG Assay Kit (IBL) according to the manufacturer's instructions. The sensitivity (lower detection limit) of the assay was 0.17 ng/ml. The inflammatory cytokines IL-6 and tumor necrosis factor alpha (TNFα) were quantified in the CS serum using an ultra-sensitive immunoassay with electrochemiluminescence detection (Meso Scale Discovery) as instructed by the manufacturer.

LRG Biomarker Assessment In Matched Subset Of Progressors And Non-Progressors

To explore the role of LRG in identifying a cohort of progressor patients, 30 patients were selected for serum analysis of LRG. An age, sex and LAVI matched subset of patients was obtained from the Non-Progressor cohort and similarly evaluated for LRG.

Quantitative Real-time PCR Analysis of LRG

Total RNA was isolated from human primary coronary artery endothelial (HCAEC) cells (Promocell) and human primary ventricular cardiac fibroblasts (HVCF) (ScienCell Research

Laboratories) using a NucleoSpin RNA II Kit (Macherey-Nagel) according to the manufacturer's instructions. HCAEC cells were confirmed by the supplier to be Von Willebrand factor positive, CD31 positive, Dil-Ac-LDL uptake positive, alpha smooth muscle actin negative, and HVCF cells were shown to be fibronectin positive. Right atrial tissue was collected from patients with multi-vessel ischemic heart disease undergoing coronary artery bypass surgery. Immediately following biopsy collection, the tissue was stored in Allprotect Tissue Reagent (Qiagen) and RNA was subsequently isolated using AllPrep DNA/RNA Mini-Kit (Qiagen) according to the manufacturer's instructions. 600 ng of RNA was reverse transcribed using SuperScript II RT (Invitrogen) prior to quantitative real-time PCR performed as previously described (5). Gene-specific primers used are as follows; LRG, 5′- GTCCTCTTGGAGCAGACAGC-3′ (forward), 5′-AGGTGGTTGACAGGAGATGG-3′ (reverse); TGFβR1, 5′-ATTGCTGGACCAGTGTGCTT-3′ (forward), 5′-AAACCTGAGCCAGAACCTGA- 3′ (reverse); alpha smooth muscle actin (ASMA), 5′-CGTTACTACTGCTGAGCGTGA-3′ (forward), 5′-AACGTTCATTTCCGATGGTG-3′ (reverse):and the housekeeping gene glyceraldehyde-3-phosphate dehydrogenase , 5′-ACAGTCAGCCGCATCTTCTT-3′ (forward), 5′-ACGACCAAATCCGTTGACTC-3′ (reverse). The samples were quantified using the delta CT method. Samples were electrophoresed on a 2% polyacrylamide gel using SybrSafe (Invitrogen) for visualization.

Immunohistochemical Analysis of LRG

For the purpose of immunohistochemical detection and localization of LRG protein within the myocardium, 3 right atrial tissue samples were collected from patients with multi-vessel ischemic heart disease undergoing coronary artery bypass surgery. Tissue specimens were immediately washed in PBS and fixed in 10% formalin, followed by paraffin embedding. Tissue sections (5 μm) were treated with 0.01M citrate buffer at pH 6 for 20 minutes at 95 ° C. prior to incubation with a rabbit anti-LRG polyclonal antibody (Atlas Antibodies) or the corresponding IgG control at an equivalent concentration. Immunoreactivity was detected with EnVision (Dako). In addition, tissue sections were stained with hematoxylin and eosin for morphological assessment of the cardiac tissue. Slides were imaged using the Aperio ScanScope digital scanner and visualized using VisionScope V10 (Aperio Technologies).

Statistical Analysis

Differences in 2D-DIGE spot volumes between low and high BNP groups were determined using Student's t-test, with p values ≦0.05 considered statistically significant.

For continuous variables, summary statistics are presented as the mean ±standard deviation (SD) or median and 25-75^{th} percentiles. Categorical variables are presented as frequencies and percentages (in parenthesis). Comparisons between the normal and elevated BNP groups were made using independent t-test, Wilcoxon's Rank Sum, or chi square test where appropriate.

The relationship between BNP and LRG was assessed using Pearson's correlation coefficient for variables that were approximately normally distributed and using log-transformation of variables with non-normal distribution. Spearman Rank order correlation was used for the analysis of non-normal tissue gene expression data. Receiver operating characteristic (ROC) curves were plotted to assess the usefulness of LRG and BNP in identifying or predicting restrictive-like filling patterns and diastolic HF in patients without systolic HF. Furthermore, we examined the ability of LRG and BNP to identify predict any HF from non-HF patients. Univariable and multivariable analyses were conducted using binary logistic regression to determine the ability of LRG to predict identify the presence of restrictive-like filling patterns or HF as the outcome variables. For multivariable analysis, the p value of the partial likelihood ratio test was used to confirm if a covariate was significant and the coefficients of the remaining variables were assessed to determine if important (>20%) changes had occurred on variable exclusion. All statistical calculations were performed using SPSS V.12 software.

For the LVDD progression study, data are presented as either mean ±SD or median (25^{th}:75^{th} percentile) for normally and non-normally distributed continuous variables respectively whereas frequencies and percentages (in parentheses) summarize categorical variables. Group comparisons were made using two-sample independent t-test, chi-squared and Mann-Whitney where appropriate. Changes are recorded as parameter values at annual review less values at baseline and are denoted by the operator Δ. Two-sided probability values are reported with p<0.05 considered as statistically significant. All logarithms presented are natural logarithms.

Results

Proteomic Analysis of CS Sera in Asymptomatic Hypertensive Patients with High and Low BNP.

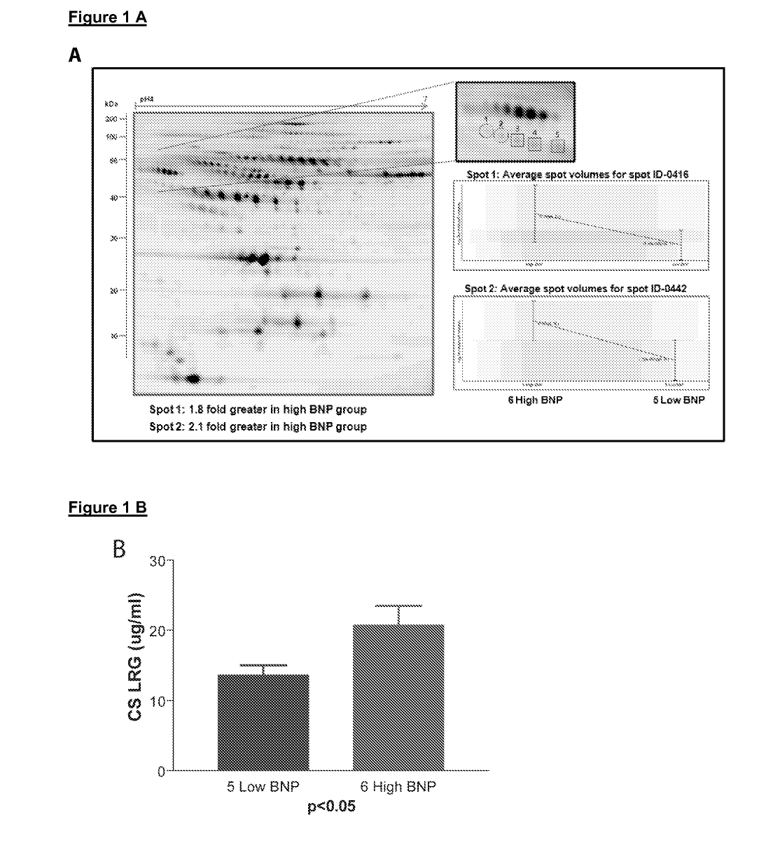

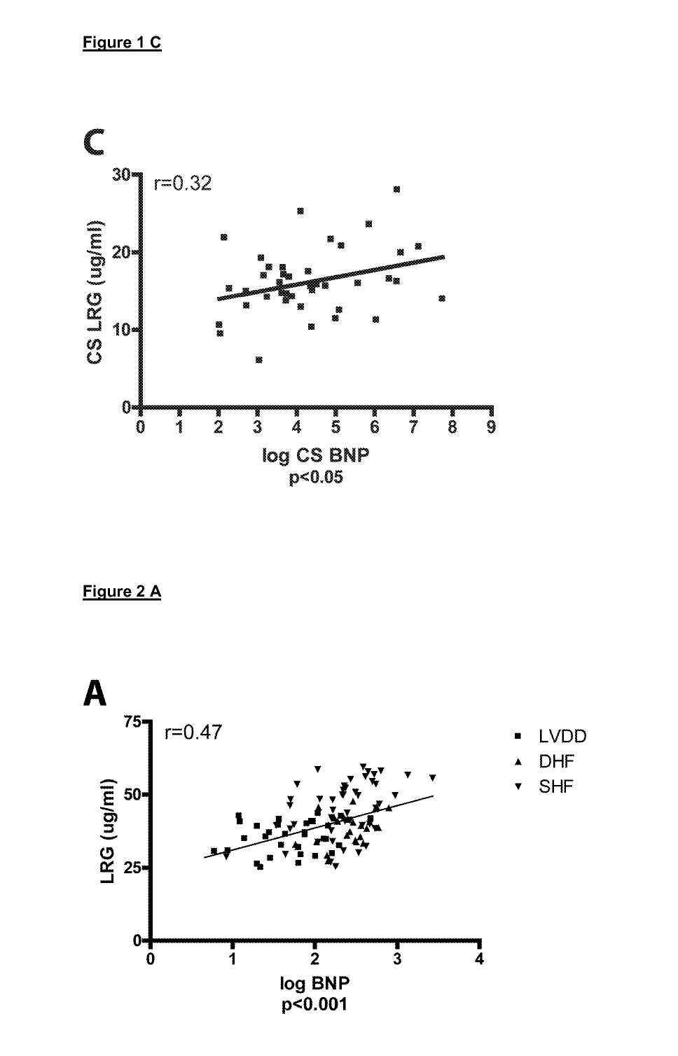

Comparison of the CS serum proteome of 11 asymptomatic hypertensive patients using 2D-DIGE and quantitative computer image analysis of 3D spot volumes revealed 949 different protein spots on the 2D gels of which 117 were differentially expressed. Two neighboring protein spots, Spot ID-0416 and Spot ID-0442, were differentially expressed in high BNP serum by 1.8 and 2.1 fold, respectively (p<0.05). The protein spots were excised from the 2D gels and identified using mass spectrometry as LRG (pl 6.5; molecular weight 38154.13 Da), FIG. 1A. The existence of LRG variants is potentially explained by post-translational glycosylation (6). Pooling of the 3D spots volumes from the two differentially expressed LRG variants revealed a significant 2 fold increase in the high BNP group. The 3 adjacent spots that were not differentially expressed (Spots 3, 4 and 5, FIG. 1A), but were in the same row as the differentially expressed LRG spots were also identified. These 3 neighboring spots were also found to be LRG. This train of LRG spots (Spots 1-5) represents variants of LRG based on the different migratory patterns through the 2D gels. The existence of LRG variants is potentially explained by post-translational glycosylation (6). Pooling of the 3D spots volumes from the two differentially expressed LRG variants revealed a significant 2 fold increase in the high BNP group.

The potential biological role of LRG prompted our focus on this protein. We verified our 2D-DIGE observation in the 11 asymptomatic hypertensive patients using ELISA (p<0.05), FIG. 1B. We further examined CS serum levels of LRG in an expanded cohort totaling 40 asymptomatic hypertensive patients. We found that LRG levels were significantly correlated with CS BNP levels higher in patients with elevated BNP (r=0.32, p<0.05), suggesting that LRG is associated with patients at risk of developing DD and HF, FIG. 1C.

TABLE 1

| | | | Fold change in |
|---|---|---|---|
| Protein name | Mol. Weight | pl | high BNP serums |
| Complement factor I precursor | 65720 | 7.72 | 6.8 increase |
| Pigment epithelium-derived factor precursor | 46340.51 | 5.971 | 4.8 increase |
| Isoform Migration stimulation factor FN70 of | 71923.84 | 6.6 | 4.8 increase |
| Fibronectin | | | |
| Serum paraoxonase/arylesterase 1 | 39747.93 | 5.084 | 4.2 increase |
| Beta-2-glycoprotein (Fib) | 38272.67 | 7.8 | 3.4 increase |
| Apolipoprotein a-iv precursor | 45397.41 | 5.282 | 3.1 increase |
| Apolipoprotein a-i precursor | 30776.66 | 5.555 | 2.6 increase |
| Leucine-rich alpha-2-glycprotein | 38154.13 | 6.5 | 2.1 increase |
| Complement C3 (Fib) | 187029.3 | 6 | 1.7 increase |
| Zinc-alpha-2-glycoprotein | 33850.91 | 5.5 | 1.7 increase |
| Tetranectin precursor | 22566.04 | 5.523 | 2.1 decrease |
| Serum amyloid P-component | 25371.13 | 6.1 | 1.7 decrease |
| Complement c1s subcomponent precursor | 76681.46 | 4.857 | 1.7 decrease |
| Vitamin d-binding protein precursor | 52915.4 | 5.319 | 1.6 decrease |
| Isoform 1 of gelsolin precursor | 85693.97 | 5.896 | 1.6 decrease |
| Transthyretin precursor | 15886.35 | 5.522 | 1.5 decrease |
| Clusterin | 52461.05 | 5.9 | 1.5 decrease |

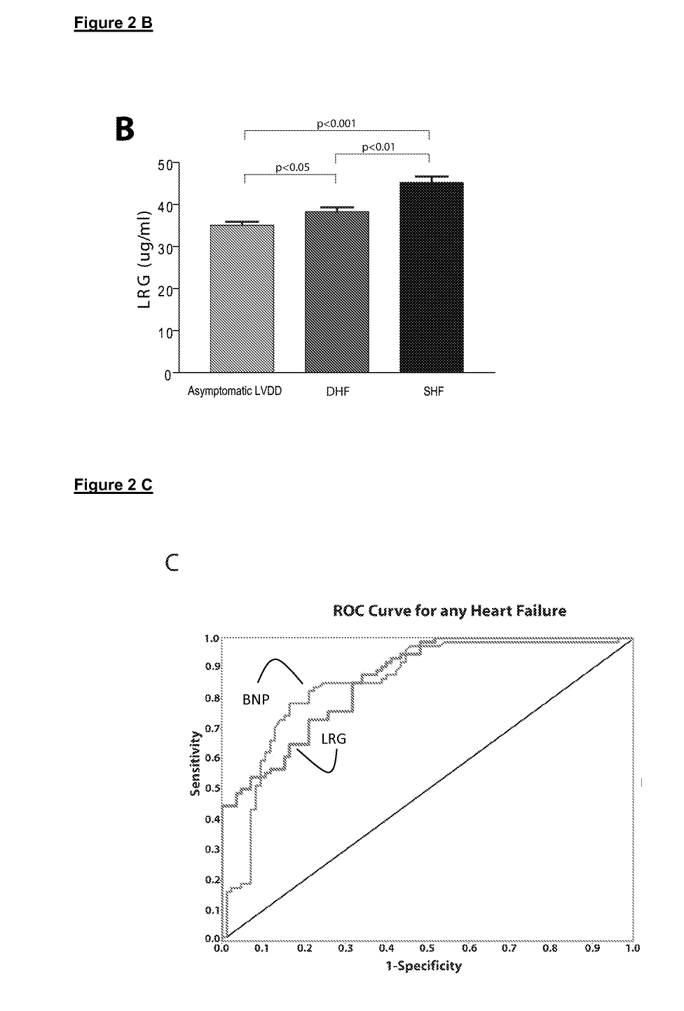

Quantification of LRG levels across the spectrum of ventricular dysfunction. Serum levels of LRG were further quantified in additional patient cohorts across the spectrum of asymptomatic LVDD, DHF, and SHF, (Table 2). LRG levels within the peripheral serum significantly correlated with BNP levels (p<0.001, r=0.47), FIG. 2A. A significant incremental increase in serum LRG levels was detected between asymptomatic LVDD and DHF (P<0.05), asymptomatic LVDD and SHF (p<0.001), DHF and SHF (p<0.01), FIG. 2B. Serum LRG correlated with Echocardiography parameters. When asymptomatic hypertensive patients, asymptomatic LVDD patients, and DHF patients were analyzed collectively, it was found that LRG significantly correlated with left ventricular mass index (p=0.01, r=0.25).

LRG Identifying/predicting Restrictive-like Filling Patterns and HF

ROC curve analyses showed that LRG and BNP were able to significantly identify patients with predictive of restrictive-like filling patterns (n=15 vs. n=83 normal or mild/moderate diastolic dysfunction, AUC 0.82, 0.85 respectively, both p<0.001) and DHF (n=26 DHF vs. N=72 no-HF, AUC 0.87, 0.81 respectively, both p<0.001). Furthermore, both LRG and BNP were significantly predictive of useful in identifying any HF in the dataset (n=73 HF vs. n=72 no-HF, AUC 0.86, 0.85 respectively, both p<0.001, FIG. 2C). Multivariable analysis showed that LRG's was significantly predictive of ability to identify any HF is independent of age, sex, creatinine and BNP (adjusted HR 1.460, 95% Cl:1.183-1.801, p<0.0001). In this model, BNP was not predictive of able to discriminate the HF population from the no HF population (adjusted HR 1.003, 95% Cl: 0.999-1.006, p=0.171). Importantly, LRG continued to be able to remained significantly identify predictive of HF when the multivariable analysis was further adjusted for beta blocker usage and ischemic events (adjusted HR 1.755, 95% Cl:1.022-3.013, p<0.05).

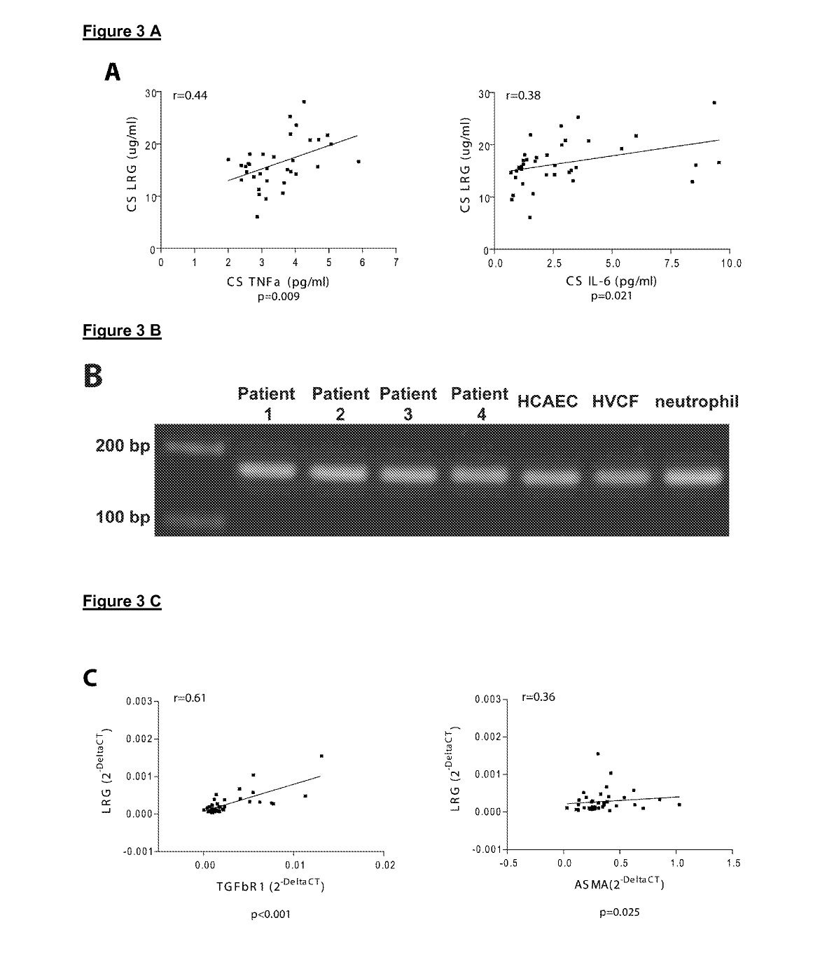

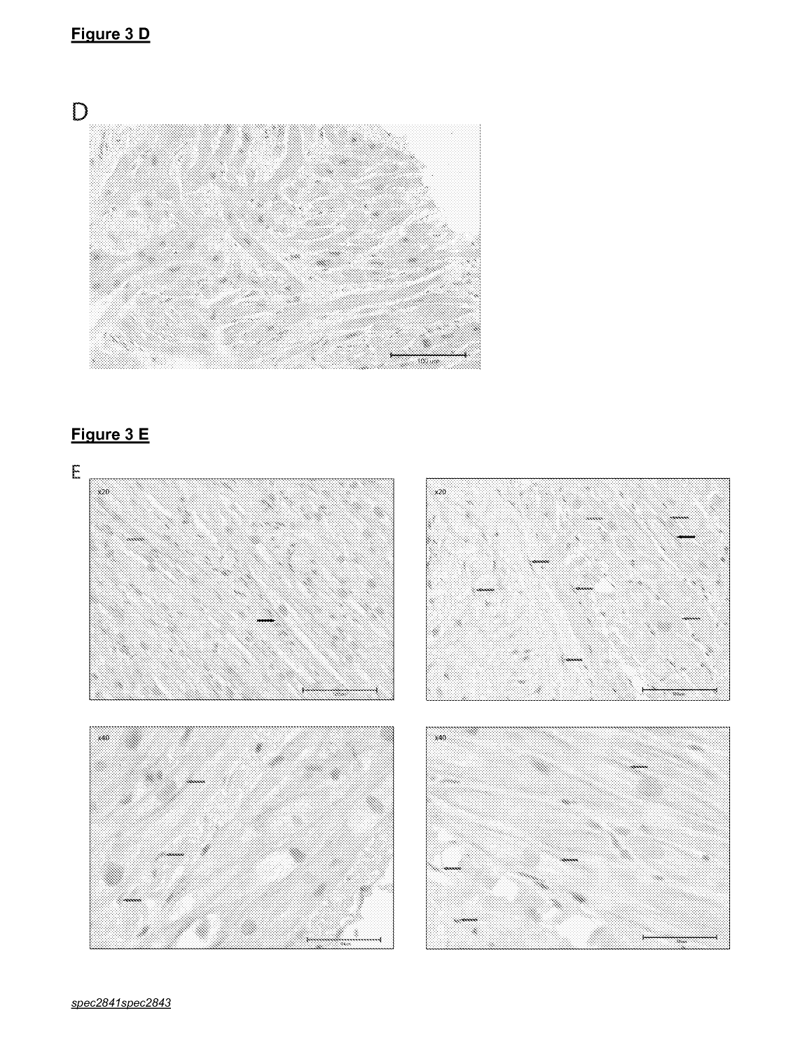

Relationship between LRG and inflammation. CS serum levels of LRG significantly correlated with the inflammatory cytokines TNFα (p=0.009, r=0.44) and IL-6 (p=0.021, r=0.38) in an asymptomatic hypertensive population, FIG. 3A.

Relationship Between Cardiac Tissue LRG and Expression of Fibrogenic Genes.

PCR analysis of LRG expression was assessed in commercially available human primary cells that originated from cardiac tissue, right atrial biopsy tissue samples, and human primary neutrophils. In vitro evidence of LRG mRNA production was detected in primary HCAEC and VHCF, FIG. 3B. Evidence of myocardial tissue being a potential source of LRG was also confirmed, FIG. 3B.

The relationship between cardiac tissue LRG, TGFβ receptor, and the myofibroblast related gene ASMA was assessed in 40 right atrial tissue samples using quantitative real-time PCR. Cardiac tissue LRG significantly correlated with both TGFβ receptor (p<0.001, r=0.61) and ASMA (p=0.025, r=0.36), FIG. 3C.

Confirmatory evidence of LRG protein being localized within the myocardium is highlighted in FIG. 3E. Examination of 3 cardiac biopsies revealed that cardiac myocytes, which are apparent as the largest cell type within the tissue section, exhibited positive immuno-staining for LRG. The distribution and intensity of LRG within the myocytes was varied, with specific cytoplasmic and membraneous expression detected. Cardiac fibroblasts, the more numerous but smaller cell type within the heart, exhibiting elongated nuclei, are also evident within FIG. 3E, and appear to be negative for LRG protein expression.

Baseline Characteristics:

Patient demographics for the study population and each subgroup are listed (Table 2). The study population involved equal numbers of males and females (n=114) with an average age of 65±10 years. A high prevalence of hypertension (68%) and hypercholesterolemia (67%) was recorded across the entire cohort of whom 18% were known to have coronary disease (via angiography) and 6% had previous myocardial infarction. The prevalence of DM was 10%.

Conversely, LRG remains unchanged in the Non-progressor population (68.4±33.1 at baseline to 67.4±28.4 ng/mL, within group difference p=0.40) whereas it increases in the Progressor population (72.0±27.8 yo 86.1±27.4 ng/mL, within group difference p=0.017, between group difference p=0.001).

TABLE 2
Baseline Characteristics of the entire Study Population

| Baseline Demographic | Entire Cohort | Progressors | Non Progressors |
|---|---|---|---|
| N | 228 | 33 (14%) | 195 (86%) |
| Age, yr | 65 ± 10 | 68 ± 8* | 64 ± 10 |
| Gender, male | 114 (50%) | 17 (52%) | 97 (50%) |
| BMI, kg/m² | 26 ± 4 | 29 ± 4 | 26 ± 4 |
| Waist Size, cm | 39 ± 4 | 39 ± 4 | 39 ± 4 |
| BSA, m² | 1.9 ± 0.2 | 1.9 ± 0.2 | 1.9 ± 0.2 |
| SBP/DBP, mmHg | 142/82 ± 18/11 | 145/82 ± 20/11 | 141/82 ± 18/11 |
| HR, bpm | 60 ± 15 | 63 ± 14 | 59 ± 15 |
| Hypertension | 154 (68%) | 25 (76%) | 129 (66%) |
| Hypercholesterolemia | 152 (67%) | 24 (73%) | 128 (66%) |
| IHD | 41 (18%) | 7 (21%) | 34 (17%) |
| Previous MI | 23 (10%) | 4 (12%) | 19 (10%) |
| Diabetes Mellitus | 25 (11%) | 4 (12%) | 21 (11%) |
| Arrhythmia | 13 (6%) | 4 (12%) | 9 (5%) |
| Stroke | 2 (1%) | 1 (3%) | 1 (1%) |
| COPD | 2 (1%) | 0 (0%) | 2 (1%) |
| Aspirin | 114 (50%) | 21 (64%) | 93 (48%) |
| Statin | 153 (67%) | 24 (73%) | 129 (66%) |
| ARB | 73 (32%) | 15 (45%) | 58 (30%) |
| Beta Blocker | 65 (29%) | 17 (52%)* | 48 (25%) |
| ACE Inhibitor | 64 (28%) | 9 (27%) | 55 (28%) |
| Calcium Channel Antagonist | 54 (24%) | 11 (33%) | 43 (22%) |
| Aldosterone Antagonist | 2 (1%) | 0 (0%) | 2 (2%) |
| Creatinine, mg/dl | 80 ± 24 | 84 ± 18 | 79 ± 25 |
| Total Cholesterol, mmol/L | 4.6 ± 1.1 | 4.2 ± 1.0 | 4.6 ± 1.1 |
| HDL, mmol/l | 1.4 ± 0.5 | 1.3 ± 0.4 | 1.4 ± 0.5 |
| LDL, mmol/l | 2.5 ± 1.0 | 2.5 ± 1.0 | 2.5 ± 1.0 |
| TG, mmol/l | 1.7 ± 1.0 | 1.8 ± 0.9 | 1.7 ± 1.0 |
| BNP, pg/ml | 18.5 (9.4:36.1) | 31.8 (17.2:50.4)* | 16.1 (8.9:33.3) |
| LVEF, % | 67 ± 9 | 68 ± 10 | 67 ± 8 |
| LVMI, g/m² | 96 ± 24 | 105 ± 27* | 95 ± 23 |
| LAVI, mls/m² | 24.9 ± 3.3 | 24.2 ± 3.4 | 25.0 ± 3.3 |
| LA Volume, mls | 47 ± 8 | 46 ± 9 | 47 ± 8 |
| E/e′ | 8.3 ± 2.7 | 8.8 ± 2.0 | 8.3 ± 2.8 |

Regarding pharmacotherapy, half of the patients were being treated with aspirin while two-thirds were on statins. Anti-hypertensive treatments included angiotensin receptor blockers (32%), diuretics (30%), beta-blockers (29%), angiotensin converting enzyme inhibitors (28%), calcium channel antagonists (24%) and aldosterone antagonists (1%). A low prevalence of concomitant pathologies was recorded in the study population: chronic obstructive airways disease (1%), stroke (1%), chronic renal failure (0%).

Subgroup Demographics:

Overall, 33 individuals showed progression of diastolic dysfunction during the study period (Table 2). Patients in this progressor subgroup were older [68±8 versus 64±10yrs], were more likely to be treated with beta-blockers [52% versus 25%], had higher levels of BNP [31.8 (17.2:50.4) versus 16.1 (8.9:33.3)] and had higher left ventricular mass indices [105±27 versus 95±23 yrs] than the non-progressor subgroup. The prevalence of hypertension and obesity was similar between both cohorts.

Changes in Parameters between Time-points:

Changes in echocardiographic parameters and BNP levels recorded at annual review compared to baseline visit are presented (Table 3). The mean change in LAVI at annual review in the progressor cohort was 6.7 ±3.8 compared to a mean change in LAVI of −0.3±3.8 mls/m² in the non-progressor cohort. Left ventricular filling pressures as assessed by E/e′ were not significantly different in progressors (0.6±3.2) versus non-progressors (0.3±2.4). Left ventricular ejection fraction was essentially unchanged for both progressors (−1±5) and non-progressors (1±8) within the study period. Similarly, BNP levels were also unchanged for both progressors [−0.2 (−11.2:10.4)] and non-progressors [0.2 (−4.8:7.2)] within the study period.

Evaluation of LRG In Matched Progressor and Non-progressor Populations:

When the matched Progressor and Non-progressor populations are compared (Table 4), the profiles and differences are similar to that of the entire study population. Furthermore, the pattern of relative echocardiography and BNP changes in the entire study population. In particular, there is no significant change in BNP over the follow-up period. This indicates that BNP does not track the progression of LVDD in this cohort.

BMI indicates body mass index; BSA, body surface area; SBP/DBP, systolic and diastolic blood pressure; HR, heart rate; IHD, ischaemic heart disease; MI, myocardial infarction; COPD, chronic obstructive airways disease; CV, cardiovascular; ARB, angiotensin II receptor blocker; ACEI, angiotensin converting enzyme inhibitor; HDL, high density lipoprotein; LDL, low density lipoprotein; TG, triglycerides; BNP, brain natriuretic peptide; LVEF, left ventricular ejection fraction; LVMI, left ventricular mass index; DT, E wave deceleration time; IVRT, isovolumetric relaxation time; LVEDD, left ventricular end-diastolic dimension; IVS, intraventricular septum; PW, posterior wall; RWT, relative wall thickness; LAVI, left atrial volume index; E/e′, ratio of mitral early diastolic flow velocity over tissue Doppler mitral annular lengthening velocity.

Values are mean ±SD, median (25^{th}:75^{th} percentile) or n (%).

*p<0.05 Progessors vs Non-progressors

TABLE 3
Differences in Echocardiographic Findings and BNP between baseline and
follow up in the entire Study Population

| Echo Parameter | Entire Cohort | Progressors | Non Progressors |
|---|---|---|---|
| Δ LVEF, % | 0 ± 7 | −1 ± 5 | 1 ± 8 |
| Δ LAVI, mls/m² | 0.7 ± 3.2 | 6.7 ± 3.8* | −0.3 ± 1.6 |
| Δ LA Volume, | 1.5 ± 5.9 | 11.7 ± 7.5* | −0.3 ± 3.2 |
| mls | | | |
| Δ E/e′ | 0.3 ± 2.5 | 0.6 ± 3.2 | 0.3 ± 2.4 |
| Δ BNP, pg/ml | 0.2 (−5.8:7.6) | −0.2 (−11.2:10.4) | 0.2 (−4.8:7.2) |

LAVI indicates left atrial volume index;
E/e′ ratio of mitral early diastolic flow velocity over tissue Doppler mitral annular lengthening velocity;
LVEF, left ventricular ejection fraction;
BNP, brain natriuretic peptide.
Values are mean ± SD or median (25^{th}:75^{th} percentile).
*p < 0.05 Progessors vs Non-Progressors

TABLE 4
Baseline Characteristics of the Progressor Sub-Study Population

| Baseline | | | |
|---|---|---|---|
| Demographic | Progressors | Non Progressors | p value |
| N | 30 | 30 | NS |
| Age, yr | 69 ± 8 | 69 ± 8 | NS |
| Gender, male | 15 (50%) | 15 (50%) | NS |
| Follow-up Duration, | 17 ± 6 | 15 ± 5 | NS |
| months | | | |
| BMI, kg/m² | 29 ± 5 | 27 ± 4 | NS |
| Waist Size, cm | 39 ± 5 | 39 ± 4 | NS |
| BSA, m² | 1.9 ± 0.2 | 1.8 ± 0.2 | NS |
| SBP/DBP, mmHg | 144/81 ± 17/10 | 138/77 ± 15/11 | NS |
| HR, bpm | 69 ± 14 | 70 ± 10 | NS |
| Hypertension | 22 (73%) | 20 (66%) | NS |
| Echocardiographic | 11 (37%) | 8 (27%) | NS |
| LVH | | | |
| Diabetes Mellitus | 3 (10%) | 5 (17%) | NS |
| Hyper- | 22 (73%) | 20 (66%) | NS |
| cholesterolemia | | | |
| Obesity | 8 (27%) | 9 (30%) | NS |
| IHD | 3 (10%) | 4 (13%) | NS |
| Previous MI | 1 (3%) | 3 (10%) | NS |
| Stroke | 1 (3%) | 0 (0%) | NS |
| COPD | 0 (0%) | 0 (0%) | NS |
| Any CV Treatment | 30 (100%) | 29 (97%) | NS |
| Anti-platelet | 18 (60%) | 21 (70%) | NS |
| Agent | | | |
| Statin | 22 (73%) | 20 (66%) | NS |
| Any | 26 (87%) | 24 (80%) | NS |
| Anti-hypertensive | | | |
| ACE Inhibitor | 6 (20%) | 10 (30%) | NS |
| ARB | 12 (40%) | 5 (17%) | NS |
| Aldosterone | 0 (0%) | 0 (0%) | NS |
| Antagonist | | | |
| Beta Blocker | 12 (40%)* | 4 (13%) | p = 0.040 |
| Alpha Blocker | 1 (3%) | 2 (6%) | NS |
| Calcium Channel | 11 (33%) | 10 (30%) | NS |
| Blocker | | | |
| Diuretic | 9 (27%) | 36 (0%) | NS |
| Creatinine, mg/dl | 84 ± 21 | 85 ± 17 | NS |
| Total Cholesterol, | 4.9 ± 1.4 | 4.5 ± 0.8 | NS |
| mmol/L | | | |
| HDL, mmol/l | 1.3 ± 0.4 | 1.2 ± 0.4 | NS |
| LDL, mmol/l | 2.8 ± 1.0 | 2.4 ± 0.7 | NS |
| TG, mmol/l | 1.8 ± 0.9 | 1.9 ± 1.1 | NS |
| BNP, pg/ml | 31.8 (9.9:63.8)* | 19.0 (5.0:50.5) | p = 0.009 |
| LRG, ng/ml | 72.0 ± 27.8 | 68.4 ± 33.1 | NS |
| LVEF, % | 67 ± 9 | 67 ± 10 | NS |
| Peak E wave, cm/s | 68 ± 17 | 64 ± 15 | NS |
| DT, ms | 182 ± 54 | 178 ± 48 | NS |
| S Wave, cm/s | 56 ± 18 | 57 ± 12 | NS |
| IVS, mm | 12 ± 2 | 11 ± 1 | NS |
| PW, mm | 10 ± 1 | 10 ± 2 | NS |
| LVMI, g/m² | 103 ± 21* | 92 ± 23 | p = 0.037 |
| LAVI, mls/m² | 23.7 ± 3.9 | 23.9 ± 3.9 | NS |
| E/e′ | 8.7 ± 2.3 | 9.2 ± 3.7 | NS |

BMI indicates body mass index;
BSA, body surface area;
SBP/DBP, systolic and diastolic blood pressure;
HR, heart rate;
LVH, Left Ventricular Hypertrophy;
IHD, ischaemic heart disease;
MI, myocardial infarction;
COPD, chronic obstructive airways disease;
CV, cardiovascular;
ACEI, angiotensin converting enzyme inhibitor;
ARB, angiotensin II receptor blocker;
HDL, high density lipoprotein;
LDL, low density lipoprotein;
TG, triglycerides;
BNP, brain natriuretic peptide;
LVEF, left ventricular ejection fraction;
DT, E wave deceleration time;
IVS, intraventricular septum;
PW, posterior wall;
LVMI, left ventricular mass index;
LAVI, left atrial volume index;
E/e′, ratio of mitral early diastolic flow velocity over tissue Doppler mitral annular lengthening velocity.
Values are mean ± SD, median (10^{th}:90^{th} percentile) or n (%).
*p < 0.05 Progessors vs Non-progressors.
Echocardiographic LVH was defined as LVMI >115 g/m² for males or >95 g/m² for females.

TABLE 5
Differences in Echocardiographic Findings, BNP and LRG between
baseline and follow up in the Progressor Sub-Study Population

| Changes in Parameters | Progressors | Non Progressors | p value |
|---|---|---|---|
| N | 30 | 30 | NS |
| Δ LVEF, % | −1 ± 6 | 0 ± 7 | p = NS |
| Δ Peak E Wave | 0 ± 12 | −2 ± 11 | p = NS |
| Velocity | | | |
| Δ DT, ms | 13 ± 44 | −8 ± 67 | p = NS |
| Δ LVMI, g/m² | −6 ± 17 | −3 ± 18 | p = NS |
| Δ LAVI, mls/m² | 7 ± 4* | 0 ± 2 | p < 0.001 |
| Δ E/e′ | 1 ± 3 | −1 ± 3 | p = 0.050 |
| Δ BNP, pg/ml | −1.1 (−24.4:35.0) | 0.9 (−26.8:37.1) | p = NS |
| Δ LRG, ng/ml | 14.11 ± 35.3 | −0.91 ± 38.1 | p = 0.001 |

LAVI indicates left atrial volume index;
E/e′ ratio of mitral early diastolic flow velocity over tissue Doppler mitral annular lengthening velocity;
LVEF, left ventricular ejection fraction;
BNP, brain natriuretic peptide.
Values are mean ± SD or median (25^{th}:75^{th} percentile).
*p < 0.05 Progessors vs Non-Progressors

Discussion

Natriuretic peptides have been shown by us and others to effectively identify various stages of DD in HHD and also to be of prognostic importance in established HF. Using BNP to stratify a population of asymptomatic hypertensive patients, we sought to identify novel biomarkers that may represent evidence of early myocardial injury which in addition, may provide insight into disease pathogenesis, susceptibility to the development of ventricular dysfunction and potentially guide novel diagnostic and therapeutic strategies. 2D-DIGE analysis of CS serum followed by mass spectrometry revealed exaggerated expression of LRG in patients with elevated BNP (≧100 pg/ml). LRG-specific ELISA validated this finding in CS sera from an extended cohort of asymptomatic hypertensive patients. We further examined CS serum levels of LRG in an expanded cohort totaling 40 asymptomatic hypertensive patients and found that LRG levels significantly correlated with CS BNP levels. In this patient cohort, LRG was also found to correlate with expression of the inflammatory cytokines IL-6 and TNFα. LRG mRNA expression was confirmed in human primary cardiac fibroblasts, primary coronary artery endothelium, and cardiac tissue biopsy samples. Correlation of LRG mRNA with TGFβR1 and ASMA expression in myocardial tissue suggests a link to fibrogenic pathways. Immunohistochemistry confirmed that LRG protein was expressed within human myocardial tissue and suggests that cardiac myocytes are the primary tissue source. Further analysis of peripheral serum from an asymptomatic LVDD, DHF, and SHF patient cohort identified LRG as a potential novel biomarker of early and sustained ventricular dysfunction and heart failure. This observation is supported by ROC curve analysis showing significant predictive power the ability of LRG to significantly identify for restrictive filling patterns and HF. In addition, multivariable modeling in this cohort shows that LRG is a stronger distinguisher predictor indicator of HF than BNP and this is independent of age, sex, creatinine, ischemia, beta blocker therapy and BNP. LRG expression was confirmed in myocardial tissue samples at both the protein and mRNA level. Correlation of LRG mRNA with TGFβR1 and ASMA expression suggests a link to fibrogenic pathways. These data indicate suggest that LRG may be a valuable marker of ventricular dysfunction from its earliest stage, and may add to our ability to diagnose and monitor patients with HF. Furthermore, with its links to inflammation and fibrogenic pathways LRG may provide insight into strategies to deploy in the into the pathogenesis earliest stages of this syndrome to halt or slow the development of ventricular dysfunction.

Although there are no data to date to indicate the precise function of LRG, the presence of a leucine at every seventh position in segments of this protein suggest the possibility of forming a leucine-zipper structure (2) which has been implicated in protein-DNA and protein-protein interaction. Also, LRG has been shown to be associated with neutrophilic differentiation (7,8) and prevention of lymphocyte apoptosis (9), while in hepatoma cell lines LRG expression has been shown to be associated with increased susceptibility to TGF-induced growth suppression (10). Some evidence suggests that LRG may be an acute phase protein. For example, its expression is induced synergistically by IL-6 and TNF in hepatocytes (11). Mass spectrometry analysis of urine from acute pediatric appendicitis patients has recently identified LRG as a potential biomarker that correlates with the severity of appendicitis (12) Further, a 9.5 fold enrichment in LRG was shown in diseased appendices which was accompanied by an 11 fold enrichment in TGFβR2. Although immunostaining localised LRG to neutrophilic focal lesions in diseased appendices, the association with TGFβR2 implies a potential overlap with TGF-driven repair mechanisms. TGFβ-induced fibrogenic responses require dimerization of TGFβ2 with TGFβR1 to enable signal transduction and downstream activation of the SMAD2/3 pathway (13). In support of a role of LRG in cardiac fibrosis we have provided evidence of LRG protein expression within the myocardium. We have also , and demonstrated a positive correlation between LRG and TGFβR1 gene expression. The association between LRG and TGF-induced fibrogenic pathways osis is further supported by a positive correlation between LRG and ASMA gene expression in myocardial tissue. Given that inflammation potentially drives myocardial injury and precedes the development of fibrosis it is noteworthy that LRG serum levels were found to correlate with those of IL-6 and TNFα in asymptomatic hypertensive patients.

The presence of LRG, with described links to inflammation in the earliest phases of ventricular dysfunction provides further supportive data to the hypothesis that inflammation may be a critical early stimulus in HHD. Indeed it may be that this proposed inflammatory stimulus may help explain the natriuretic response seen in these patients. As a cardioprotective protein BNP likely represents a response to early injury. The association between LRG and inflammation as well as echocardiographic parameters of cardiac structure (LVMI) may reflect a role for LRG in the myocardial response to early injury and the initiation of tissue repair processes. It is the downstream effects of inflammation that likely initiate interstitial disease within the myocardium which we and others have shown to be a critical pathophysiological process in the development of DD. Indeed it may be that this proposed inflammatory stimulus may help explain the natriuretic response seen in these patients. As a cardioprotective protein BNP likely represents a response to early injury.

Our data also demonstrate that LRG may be a valuable biomarker later in the syndrome of ventricular dysfunction when HF is present. We have defined a stepwise increase in LRG levels comparing patients with asymptomatic HHD, those with HF and preserved systolic function and those with systolic dysfunction HF. The stimulus for LRG in these later phases of ventricular dysfunction is unclear but it is reasonable to hypothesize that it could again be linked to the low grade inflammation observed in this syndrome. Natriuretic peptides have some limitations in the diagnosis of HF, such as significant biological variability and dependence on non-cardiac influences such as beta blocker use, BMI, renal function and age. Therefore, identification of other biomarkers and their use in isolation or with natriuretic peptides may provide a more accurate detection of sub-clinical disease and indeed prediction of progression, outcome or response to treatment. Although there is a statistically significant increase in serum LRG levels across patient groups it should be noted that the magnitude of the increase is subtle. Although further work is required to evaluate the performance of LRG as a diagnostic and prognostic biomarker in DD and HF, however the present data should encourage such work given the comparable and independent diagnostic predictive power of LRG versus BNP, the current guideline standard for biochemical diagnosis of HF(14). Further work on possible post-translational modifications of LRG may also increase the utility of this marker. The presence of LRG in five distinct spots on our 2D gels suggests the existence of multiple forms of LRG. This is supported by previous studies that have shown an increase in LRG fucosylation, mannosylation, and sialylation in pancreatic cancer (6). Even though we show a clear association of LRG with HF it is possible that identification of specific LRG glycosylation patterns may strengthen this observation. The ELISA used in this study potentially recognizes all forms of LRG therefore the signal may be diluted by the variants that are not differentially expressed. The development of an LRG variant-specific ELISA may greatly improve the diagnostic power of this marker.

The results presented here provide a detailed examination of the natural history of LVDD progression in a population with definite cardiovascular risk factors. A high annual incidence of progressive LVDD (14%) was evident amongst patients with cardiovascular risk factors. No significant gender difference or difference in rates of hypertension, DM, coronary artery disease, obesity or smoking was noted in the progressor cohort versus the non-progressor cohort. Risk factors for progression of LVDD identified in this study included increased age, beta-blocker usage, left ventricular mass index and BNP level.

BNP is a widely used, well-established risk predictor known to discriminate individuals at increased risk of heart failure and death which has been linked with echocardiographic evidence of diastolic dysfunction (15). We found that BNP levels were greater at baseline and at annual review in the progressor group compared to the non-progessor group.

However, potential confounders in this regard include increased age and a higher use of beta-blocker therapy in the progressor group, both known to be independently associated with increased BNP levels. Importantly, we found that changes in BNP do not track with LVDD progression. Consistent with these findings, previous studies have suggested that performance characteristics of BNP are typically suboptimal for identifying LV remodeling phenotypes unless high-risk individuals are targeted (16,17). LRG, on the other hand tracks the progression of LVDD in the subset analysis.

The clinical utility of identifying individuals with evidence of progressive LVDD ultimately relies upon the availability of a proven intervention. However, an ability to identify higher risk patients of itself is also a useful initial goal. For instance, this subgroup would likely obtain benefit from more attentive blood pressure and other risk factor control. Equally, higher risk individuals represent the most suitable candidates for interventional trials evaluating novel treatments to prevent HF and subsequently would likely derive most benefit from any therapeutic approach that might evolve. Unlike BNP which has been advocated as a biomarker for the identification and monitoring of LVDD, LRG tracks progression of LVDD in an asymptomatic hypertensive cohort and this suggests that LRG may have a role in identification of patients at high risk for progression to DHF and HFPEFIn summary, these novel findings linking LRG with ventricular dysfunction and HF suggest that this protein may have value as a biomarker in this syndrome. In particular, given its expression early in the natural history of this syndrome, LRG may have a role in the identification of those at risk for progression of disease and possibly even provide an insight into the relevant pathophysiological signals at play.

The identification of a number of proteins differentially expressed in patients with asymptomatic hypertensive disease allows the construction of a panel of biomarkers which can be used in the diagnosis and treatment of cardiovascular disease.

The words “comprises/comprising” and the words “having/including” when used herein with reference to the present invention are used to specify the presence of stated features, integers, steps or components but does not preclude the presence or addition of one or more other features, integers, steps, components or groups thereof.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

References

1. Haupt H, Baudner S. [Isolation and characterization of an unknown, leucine-rich 3.1- S-alpha2-glycoprotein from human serum (author's transl)]. Hoppe Seylers Z Physiol Chem 1977;358:639-46.

2. Takahashi N, Takahashi Y, Putnam F W. Periodicity of leucine and tandem repetition of a 24-amino acid segment in the primary structure of leucine-rich alpha 2-glycoprotein of human serum. Proc Natl Acad Sci U S A 1985;82:1906-10.

3. Jaubert M P, Armero S, Bonello L, Nicoud A, Sbragia P, Paganelli F, Argues S. Predictors of B-type natriuretic peptide and left atrial volume index in patients with preserved left ventricular systolic function: an echocardiographic-catheterization study. Arch Cardiovasc Dis. 2010;103(1):3-9.

4. Milan A, Caserta M A, Dematteis A, Naso D, Pertusio A, Magnino C, Puglisi E, Rabbia F, Pandian N G, Mulatero P, Veglio F. Blood pressure levels, left ventricular mass and function are correlated with left atrial volume in mild to moderate hypertensive patients. J Hum Hypertens. 2009;23(11):743-750.

5. Watson J A, Watson C J, McCrohan A M, et al. Generation of an epigenetic signature by chronic hypoxia in prostate cells. Hum Mol Genet 2009.

6. Patwa T H, Zhao J, Anderson M A, Simeone D M, Lubman D M. Screening of glycosylation patterns in serum using natural glycoprotein microarrays and multi-lectin fluorescence detection. Anal Chem. 2006; 78:6411-6421.

7. O'Donnell L C, Druhan L J, Avalos B R. Molecular characterization and expression analysis of leucine-rich alpha2-glycoprotein, a novel marker of granulocytic differentiation. J Leukoc Biol 2002;72:478-85.

8. Ai J, Druhan L J, Hunter M G, Loveland M J, Avalos B R. LRG-accelerated differentiation defines unique G-CSFR signaling pathways downstream of PU.1 and C/EBPepsilon that modulate neutrophil activation. J Leukoc Biol 2008;83:1277-85.

9. Codina R, Vanasse A, Kelekar A, Vezys V, Jemmerson R. Cytochrome c-induced lymphocyte death from the outside in: inhibition by serum leucine-rich alpha-2-glycoprotein-1. Apoptosis 2009.

10. Sun D, Kar S, Carr B I. Differentially expressed genes in TGF-beta 1 sensitive and resistant human hepatoma cells. Cancer Lett 1995;89:73-9.

11. Shirai R, Hirano F, Ohkura N, Ikeda K, Inoue S. Up-regulation of the expression of leucine-rich alpha(2)-glycoprotein in hepatocytes by the mediators of acute-phase response. Biochem Biophys Res Commun 2009;382:776-9.

12. Kentsis A, Lin Y Y, Kurek K, et al. Discovery and Validation of Urine Markers of Acute Pediatric Appendicitis Using High-Accuracy Mass Spectrometry. Ann Emerg Med 2009.

13. Gauldie J, Bonniaud P, Sime P, Ask K, Kolb M. TGF-beta, Smad3 and the process of progressive fibrosis. Biochem Soc Trans 2007;35:661-4.

14. Dickstein K, Cohen-Solal A, Filippatos G, et al. ESC guidelines for the diagnosis and treatment of acute and chronic heart failure 2008: the Task Force for the diagnosis and treatment of acute and chronic heart failure 2008 of the European Society of Cardiology. Developed in collaboration with the Heart Failure Association of the ESC (HFA) and endorsed by the European Society of Intensive Care Medicine (ESICM). Eur J Heart Fail 2008;10:933-89.

15. Martos R, Baugh J, Ledwidge M, O'Loughlin C, Conlon C, Patle A, Donnelly S C, McDonald K. Diastolic heart failure: evidence of increased myocardial collagen turnover linked to diastolic dysfunction. Circulation. 2007;115(7):888-895.

16. Lee D S, Wang T J, Vasan R S. Screening for ventricular remodeling. Curr Heart Fail Rep. 2006;3(1):5-13.

17. O'Hanlon R, O'Shea P, Ledwidge M, O'Loughlin C, Lange S, Conlon C, Phelan D, Cunningham S, McDonald K. The biologic variability of B-type natriuretic peptide and N-terminal pro-B-type natriuretic peptide in stable heart failure patients. J Card Fail. 2007;13(1):50-55.

18. Byrne J C, Downes M R, O'Donoghue N, Fitzpatrick J M, Dunn M J, Watson R W. Fasting status as a consideration for human serum collection and preparation prior to depletion and analysis. Electrophoresis 2008;29:3055-9.

1. (canceled)

2. (canceled)

3. A method of determining the risk of developing cardiovascular disease in a patient comprising taking a blood sample from a patient and determining the level of leucine-rich alpha-2-glycoprotein (LRG), in the sample, an elevated or reduced level of LRG, in comparison to a control, indicating a pre-disposition to develop cardiovascular disease.

4. A method as claimed in claim 3 further comprising determining the level of at least one protein selected from the group comprising complement factor i precursor, pigment epithelium—derived factor precursor, serum paraoxonase/arylesterase 1, Beta-2-glycoprotein (Fib), apolipoprotein a-iv precursor, apolipoprotein a-i precursor, Complement C3 (Fib), tetranectin precursor. Serum amyloid P-component, complement c1s subcomponent precursor, vitamin d-binding protein precursor, isoform 1 of gelsolin precursor, Isoform Migration stimulation factor FN70 of Fibronectin, Zinc-alpha-2-glycoprotein, Clusterin and transthyretin precursor, an elevated or reduced level of which, in comparison to a control, indicating a pre-disposition to develop cardiovascular disease.

5. A method as claimed in claim 4 comprising determining the level of at least 3 proteins from the group.

6. A method as claimed in claim 4 or 5 comprising determining the level of at least 5 proteins from the group.

7. A method as claimed in claim 3 wherein the blood sample is a coronary sinus or peripheral blood sample.

8. A diagnostic assay for determination of the risk of developing cardiovascular disease in a patient comprising an antibody against leucine-rich alpha-2-glycoprotein (LRG), or a nucleotide probe for LRG or a portion thereof, the probe being DNA, RNA or cDNA.

9. An assay as claimed in claim 8 further comprising an antibody against at least one protein selected from the group comprising complement factor i precursor, pigment epithelium—derived factor precursor, serum paraoxonase/arylesterase 1. Beta-2-glycoprotein (Fib), apolipoprotein a-iv precursor, apolipoprotein a-i precursor, Complement C3 (Fib), tetranectin precursor, Serum amyloid P-component, complement c1s subcomponent precursor, vitamin d-binding protein precursor, isoform 1 of gelsolin precursor, Isoform Migration stimulation factor FN70 of Fibronectin, Zinc-alpha-2-glycoprotein, Clusterin and transthyretin precursor or a nucleotide probe for at least one protein selected from the group or a portion thereof, the probe being DNA, RNA or cDNA.

10. An assay as claimed in claim 9 comprising determining the level of at least 3 proteins from the group.

11. An assay as claimed in claim 10 comprising determining the level of at least 5 proteins from the group.

12. An assay as claimed in any one of claims 9 to 11 selected from a real-time PCR assay, a microarray assay, a histochemical assay or an immunological assay.

13. A method of identifying a therapeutic agent capable of preventing or treating cardiovascular disease, comprising testing the ability of the potential therapeutic agent to reduce or enhance the expression of leucine-rich alpha-2-glycoprotein (LRG) in a cell or cell line, or an animal or human test subject.

14. A method as claimed in claim 13 further comprising testing the ability of the potential therapeutic agent to reduce or enhance the expression of at least one protein selected from the group comprising complement factor i precursor, pigment epithelium—derived factor precursor, serum paraoxonase/arylesterase 1, Beta-2-glycoprotein (Fib), apolipoprotein a-iv precursor, apolipoprotein a-i precursor, Complement C3 (Fib), tetranectin precursor, Serum amyloid P-component, complement c1s subcomponent precursor, vitamin d-binding protein precursor, isoform 1 of gelsolin precursor, Isoform Migration stimulation factor FN70 of Fibronectin, Zinc-alpha-2-glycoprotein, Clusterin and transthyretin precursor in a cell or cell line, or an animal or human test subject.

15. A method of identifying a therapeutic agent capable of preventing or treating cardiovascular disease, comprising contacting leucine-rich alpha-2-glycoprotein (LRG), with a putative therapeutic agent and determining if the agent modulates the activity of the protein when compared with a control.

16. A method as claimed in claim 15 further comprising contacting at least one protein selected from the group comprising complement factor i precursor, pigment epithelium derived factor precursor, serum paraoxonase/arylesterase 1, Beta-2-glycoprotein (Fib), apolipoprotein a-iv precursor, apolipoprotein a-i precursor, Complement C3 (Fib), tetranectin precursor, Serum amyloid P-component, complement c1s subcomponent precursor, vitamin d-binding protein precursor, isoform 1 of gelsolin precursor, Isoform Migration stimulation factor FN70 of Fibronectin, Zinc-alpha-2-glycoprotein, Clusterin and transthyretin precursor with a putative therapeutic agent and determining if the agent modulates the activity of the protein when compared with a control.

17. A method of prevention or treatment of cardiovascular disease comprising administering to a patient in need of such treatment, an inhibitor of or an agent which can silence leucine-rich alpha-2-glycoprotein (LRG).

18. A method as claimed in claim 15 further comprising administering at least one protein selected from the group comprising complement factor i precursor, pigment epithelium derived factor precursor, serum paraoxonase/arylesterase 1. Beta-2-glycoprotein (Fib), apolipoprotein a-iv precursor, apolipoprotein a-i precursor, Complement C3 (Fib), tetranectin precursor, Serum amyloid P-component, complement c1s subcomponent precursor, vitamin d-binding protein precursor, isoform 1 of gelsolin precursor, Isoform Migration stimulation factor FN70 of Fibronectin, Zinc-alpha-2-glycoprotein, Clusterin and transthyretin precursor.

19. A solid support onto which leucine-rich alpha-2-glycoprotein (LRG), or antibodies raised against them, or nucleic acid probes for the proteins, have been fixed.

20. A solid support onto which at least one protein selected from the group comprising complement factor i precursor, pigment epithelium—derived factor precursor, serum paraoxonase/arylesterase 1, Beta-2-glycoprotein (Fib), apolipoprotein a-iv precursor, apolipoprotein a-i precursor, Complement C3 (Fib), tetranectin precursor. Serum amyloid P-component, complement c1s subcomponent precursor, vitamin d-binding protein precursor, isoform 1 of gelsolin precursor, Isoform Migration stimulation factor FN70 of Fibronectin. Zinc-alpha-2-glycoprotein, Clusterin and transthyretin precursor or antibodies raised against them, or nucleic acid probes for the proteins, have been fixed.

21-26. (canceled)
